(19) United States
(12) Patent Application Publication
(10) Pub. No.: US 2018/0027949 A1
(43) Pub. Date: Feb. 1, 2018

(54) INTEGRATED UNDER-EYE MASK AND MAKEUP CAPTURE DEVICE

(71) Applicant: L'OREAL, Paris (FR)

(72) Inventors: Kelly Marie George, Denville, NJ (US); Kruti Shah, Clifton, NJ (US); Angelike A. Galdi, Westfield, NJ (US); Michelle Salese, South Plainfield, NJ (US)

(21) Appl. No.: 15/223,340

(22) Filed: Jul. 29, 2016

(51) Int. Cl. A45D 40/30; A61K 8/81; A61Q 19/00; A61Q 19/08; A45D 44/00; A61K 8/34; A61K 8/49; A61K 8/73; A61K 8/365; A61K 8/02; A61Q 1/02

(52) U.S. Cl. CPC A45D 40/30; A61K 8/0212; A61K 8/8111; A61K 8/8147; A61Q 19/007; A61Q 19/08; A61Q 1/02; A61K 8/345; A61K 8/4926; A61K 8/735; A61K 8/365; A45D 44/002; A61K 2800/87; A61K 2800/524

(57) ABSTRACT

The present disclosure relates to an integrated under-eye mask and makeup capture device. The device captures makeup fallout generated during application of makeup to the area around the eyes, and thereby prevents it from contaminating the area directly beneath the eye. The device simultaneously acts as an under-eye mask that delivers a dermatological composition comprising one or more skin active ingredients to the skin. The device comprises a deformable sheet-like substrate having a dry top surface for capturing makeup fallout, a moist bottom surface for application to the skin, and an upper edge that fits under an eye of a user, wherein the bottom moist surface is impregnated with a dermatological composition comprising one or more skin-active ingredients.

### FIELD OF THE DISCLOSURE

The present disclosure relates to an integrated under-eye mask and makeup capture device. The device captures makeup fallout generated during application of makeup to the area around the eyes, and thereby prevents it from contaminating the area directly beneath the eye. The device simultaneously acts as an under-eye mask that delivers a dermatological composition comprising one or more skin active ingredients to the skin.

### BACKGROUND

Civilizations have used cosmetics for centuries to enhance beauty, in religious rituals, and to promote good health. Often, individuals desire the body, and in particular, the face, to look youthful and vibrant. Therefore, application of makeup, lotions, and creams to the face is common. For example, night creams, moisturizing products, and face masks are commonly used to promote and maintain skin health.

Dark circles and puffiness under the eyes are common concerns, especially for those working long hours unable to achieve optimal sleep. Some individuals complain of “raccoon eyes,” which may be attributable to having extremely thin skin around the eyes. This results from the purple-blue color of underlying blood vessels appearing through the thin and somewhat transparent skin. Certain eye masks and cosmetic creams can help firm and tone the under-eye area, especially when formulated with hydrating and plumping ingredients.

Makeup is additionally used to enhance and manipulate the appearance of the face. Although face masks, night creams, and moisturizers can contribute a great deal to the healthy appearance of skin, makeup is an additional tool used to cover or smooth aberrations of the skin, to enhance or draw attention to certain features, and to add color and contrast to the eyes, lips, etc. One vexing problem often encountered during the application of eye makeup is the unwanted contamination of the area under the eyes. As makeup is applied to the area around the eyes, powder and/or particulate material (“makeup fallout”) inadvertently falls downwardly to the area beneath the eyes. The application of mascara can also lead to unwanted under-eye smudging or flaking. The makeup fallout is often a dark or vivid color, which is characteristic of most eye makeup because it is intended to provide contrast from the subtle, more neutral colors of the face. Even a small amount of makeup fallout on the skin beneath the user's eye can discolor the skin and require removal (which can lead to irritation and redness of the delicate under-eye area) and reapplication of more makeup to cover the discoloration.

### SUMMARY OF THE DISCLOSURE

The present disclosure relates to a device that can be applied to the face directly under the eye of a user to protect the underlying skin from makeup fallout generated while applying makeup to the area around the eyes. The device captures the loose makeup particles that inevitably fall during the makeup application process. In addition to acting as a physical barrier that protects the area of the face directly beneath the eyes, the device is unique because its outer surface typically includes a material that attracts or securely binds the makeup particles. The outer surface is electrostatically charged. By sequestering the makeup particles and protecting the area under the eye, the device simplifies the overall makeup process. The area under the eye does not become contaminated with makeup fallout and users no longer need to clean or reapply makeup to this area after making-up the area around the eyes.

The device is also unique in that it simultaneously functions as an under-eye mask. The device has a moist underside (or bottom layer) that contains a dermatological composition having one or more skin-active ingredients. During use, the moist underside is applied to the skin so that the dermatological composition comes into contact with the skin. The device is made of a deformable sheet-like substrate, optionally containing multiple layers. The deformable sheet-like nature of the device allows it to hug the small contours under the eye for better application and penetration of the skin-active ingredient(s). The facial mask element of the device is useful for hydrating the skin and providing an immediate radiance boost for a plumper, fresher, and brighter look. Depending on the skin-active ingredient(s) incorporated into the dermatological composition, the under-eye mask can improve eye contour, minimize skin irregularities, and impart shine to the skin so it appears more uniform, radiant, and fresh. In some cases, the eye mask provides a tightening effect on the skin, and may stimulate new cellular growth and renewal.

More specifically, the instant disclosure is directed to an integrated under-eye mask and makeup capture device comprising: a deformable sheet-like substrate having a dry top surface for capturing makeup fallout, a moist bottom surface for application to the skin, and an upper edge to fit under an eye of a user. The bottom moist surface is impregnated with a dermatological composition comprising one or more skin-active ingredients. The deformable sheet-like substrate may comprise a single layer or multiple layers. For example, the dry top surface maybe a part of a dry surface layer and the bottom moist surface maybe part of a bottom surface layer. The top surface and the bottom surface layer may be separated by one or more additional layers, e.g., an adhesive layer, a water resistant layer, etc.

The dermatological composition is often aqueous based, but is not required to be aqueous based. In some cases, the dermatological composition comprises one or more skin-active ingredients, one or more preservatives, and one or more solvents. Additionally, the dermatological composition may optionally include one or more polyols. The dermatological composition is infused or impregnated into at least part of the bottom of the device so that when the bottom surface comes into contact with the skin the one or more active ingredients can come into contact with the skin.

The instant disclosure further relates to methods of using the integrated under-eye mask and makeup capture device. For instance, the methods include treating skin under an eye while simultaneously capturing makeup fallout comprising applying the integrated under-eye mask and makeup capture device disclosed herein to the area under the eye and applying makeup to an area around the eye. Furthermore, the instant disclosure relates to methods for hydrating the skin, reducing the appearance of wrinkles in the skin, reducing the appearance of dark circles or discoloration of the skin, improving shine of the skin, reducing puffiness of the skin, improving the elasticity of the skin, and to imparting a feeling of freshness to the skin and optionally to the eyes.

### BRIEF DESCRIPTION OF THE DRAWINGS

Implementations of the present technology will now be described, by way of example only, with reference to the attached figures, wherein:

FIG. 1 is a perspective view of an integrated under-eye mask and makeup capture device applied to the face of a user;

FIG. 2 is a magnified, front-view of the integrated under-eye mask applied under the eye of a user;

FIG. 3 is a side view of an integrated under-eye mask and makeup capture device comprising three layers;

FIG. 4 is a side view of an integrated under-eye mask and makeup capture device comprising four layers; and

FIG. 5 is a close-up view of the electrostatically charged dry top surface collecting makeup fallout.

It should be understood that the various aspects are not limited to the arrangements and instrumentality shown in the drawings.

### DETAILED DESCRIPTION OF THE DISCLOSURE

The present disclosure relates generally to an integrated under-eye mask and makeup capture device comprising:

a deformable sheet-like substrate having:
a dry top surface for capturing makeup fallout;
a moist bottom surface for application to the skin; and
an upper edge to fit under an eye of a user;

wherein the bottom moist surface is impregnated with a dermatological composition comprising one or more skin-active ingredients.

The deformable sheet-like substrate may be a single layer of material or may comprise multiple layers, for example, at least two, three, four, or more layers. In some cases, the deformable sheet-like substrate comprises at least two layers and the dry top surface is formed on a separate layer than the moist bottom surface. In other words, one layer may be a moist layer for application to the users face and another layer maybe a dry top layer for capturing makeup fallout. The two separate layers may be made of the same material or different material, and in some case internal layers may be included. For example, an internal liquid resistant layer or a waterproof layer may be included to prevent the dermatological composition from the bottom surface of the device from absorbing into the top dry surface of the device. Additionally, one or more internal adhesive layers may be included that bind to adjacent layers and hold the layers of the deformable sheet-like substrate together.

The deformable sheet-like substrate and/or the various layers of the deformable sheet-like substrate may be manufactured from a variety of materials, so long as the materials are sufficiently deformable. For example, a fiber sheet such as non-woven sheet, woven sheet, or knitted sheet may be used. Sheet-material that is absorbent is particularly useful in forming the bottom moist layer, which carries the dermatological composition. For example deformable wood fiber or other plant-based fiber sheets may be used. In some cases, a stretchable sheet, such as a stretchable fiber sheet, is useful to provide a tight feeling on the face during use. Non-limiting examples of materials of a pressed nature that are often deformable include bond paper, rag paper, reed paper, wood paper, vegetable paper, fruit paper, tissue paper, rice paper, parchment, vellum, papyrus, paperterie, paper leaf, paper foil, paper fiber, paper wafer, rubber sheet, plastic sheet, acetate sheet, and metal foil.

The top surface or top layer of the integrated under-eye mask and makeup capture device may be electrostatically charged so that the makeup fallout is attracted to the top surface or top layer. The material of the top surface or top layer may be treated to become electrostatically charged or may be made of material that is already electrostatically charged. For example, the top surface or top layer of the integrated under-eye mask and makeup capture device comprises electrostatic fibers, e.g., polypropylene and acrylic fibers, or triboelectric fibers. Often, electrostatic fibers are made out of tiny polyester fibers. The fibers are either manufacture very thin or are normal diameter fibers that have been “brushed,” which means that they have been fractured to make a tiny bundle of fiberettes (similar to “split ends”). The result is a very soft feel. The electrostatic nature of the top surface allows the surface to attract and sequester particles of makeup (makeup fallout), without the need for solvents, chemicals, or water.

At least a portion of the bottom moist surface or bottom moist layer of the integrated under-eye mask and makeup capture device is applied to the face under the eye(s) of the user. The moist nature of the surface allows the device to adhere to the skin without the need for adhesive, glue, cement, or the like. An adhesive (or glue, cement, etc.) used for attaching the device to the skin of the face can interfere with the ability of the dermatological composition to contact and deliver one or more skin-active ingredients to the skin. Furthermore, the adhesive (or glue, cement, etc.) can be irritating to the skin and difficult to remove. Residual adhesive (or glue, cement, etc.) may remain on the face after removal of the device and the skin may be pulled, stretched, or even damaged when detaching the device from the skin. Therefore, typically, the moist nature of the bottom surface of the device is usually sufficient to hold the device onto the face during use. Note that the backside of the bottom moist surface (the side that does not come into contact with the user's face) may include an adhesive (or glue, cement, etc.) for attachment to additional layers or materials of the device.

The device has an upper edge to fit under the eye of a user. The upper edge is often concave in order to outline the shape of the eye. This allows the device to be applied close to the bottom edge of the eye in order to maximize protective coverage.

The dermatological composition comprises one or more skin-active ingredient. The dermatological composition may be aqueous or anhydrous. In some cases, the dermatological composition may be an emulsion and in some cases the dermatological composition may be a dispersion, for example, an aqueous dispersion. A variety of dermatological compositions comprising various skin-active ingredients can be used with the integrated under-eye mask and makeup capture device. In some cases, the dermatological composition comprises: (a) one or more skin-active ingredients; (b) optionally, one or more polyols; (c) one or more preservatives; and (c) one or more solvents.

Non-limiting examples of skin-active ingredients include wrinkle reducing ingredients, skin whitening ingredients, moisturizing ingredients, keratolytic agents, desquamating agents, keratinocyte proliferation enhancers, collagenase inhibitors, elastase inhibitors, anti-inflammatory agents, steroids, anti-acne agents, and antioxidants. More specifically, non-limiting examples of particular skin-active ingredients include sodium hyaluronic acid, niacinamide, a peptide, linseed extract, a rice protein, caffeine, hesperetin laurate, ceramides (e.g., ceramide-3 and/or ceramide-EOP), cholesterol, proxylane, and ascorbyl glucoside. In some cases, the dermatological compositions comprises one or more (or all) of niacinamide, lactic acid, and sodium hyaluronate.

The total amount of the one or more skin active ingredients in the dermatological composition may be about 0.1 to about 25 wt. %, about 0.1 to about 20 wt. %, about 0.1 to about 15 wt. %, about 0.1 to about 10 wt. %, about 0.1 to about 5 wt. %, about 0.5 to about 25 wt. %, about 0.5 to about 20 wt. %, about 0.5 to about 15 wt. %, about 0.5 to about 10 wt. %, about 0.5 to about 5 wt. %, about 1 to about 25 wt. %, about 1 to about 20 wt. %, about 1 to about 15 wt. %, about 1 to about 10 wt. %, or about 1 to about 5 wt. %, wherein the weight percent is based on the total weight of the dermatological composition.

The dermatological composition of the integrated under-eye mask and makeup capture device may optionally include one or more polyols. The term “polyol” should be understood as meaning, within the meaning of the present disclosure, an organic molecule comprising at least two free hydroxyl groups. In some cases, the one or more polyols is/are selected from the group consisting of C₂-C₃₂ polyols. Typically, the one or more polyols are liquid at ambient temperature (25° C.). Suitable poloyls may be saturated or unsaturated and linear, branched or cyclic alkyl, carrying on the alkyl chain at least two —OH functional groups, at least three —OH functional groups, at least four —OH functional groups, or more. The one or more polyols may have from 2 to 32 carbon atoms, from 3 to 16 carbon atoms, or from 3 to 12 carbon atoms.

Among the polyols that may be used in the dermatological composition, mention may be made of glycerol, propylene glycol, butylene glycol, hexylene glycol, polyethylene glycols such as PEG-8, and dipropylene glycol, and mixtures thereof. In some cases, the polyol is one or both of glycerol and butylene glycol. Additionally, in some cases, the dermatological composition comprises at least glycerol, and optionally one or more polyols other than glycerol.

The total amount of the one or more polyols may be about 1 to about 50 wt. %, about 1 to about 30 wt. %, about 1 to about 20 wt. %, about 1 to about 15 wt. %, about 1 to about 10 wt. %, or about 1 to about 5 wt. %, wherein the weight percent is based on the total weight of the dermatological composition.

Typically, the dermatological composition comprises one or more preservatives. Non-limiting examples of useful preservatives include ethanol, polyvinyl alcohol, phenoxyethanol, benzyl alcohol, salicylic acid, sodium benzoate, benzoic acid, caprylyl glycol, methyl paraben, propyl paraben, ethylhexylglycerin, 1,3-propanediol, cholorphensin, methylchloroisothiazolinone, methylisothiazolinone, benzalkonium chloride, polyaminopropyl biguanide, and glyceryrrihia glabra. In some cases, the dermatological composition comprises one or more (or all) of chlorphensesin, phenoxyethanol, sodium benzoate, and glyceryrrihia glabra.

The total amount of the one or more preservatives in the dermatological composition is typically about 0.01 to about 10 wt. %, about 0.01 to about 5 wt. %, about 0.01 to about 2 wt. %, 0.01 to about 1 wt. %, about 0.05 to about 10 wt. %, about 0.05 to about 5 wt. %, about 0.05 to about 2 wt. %, about 0.05 to about 1 wt. %, about 0.1 to about 10 wt. %, about 0.1 to about 5 wt. %, about 0.1 to about 2 wt. %, or about 0.1 to about 1 wt. %, wherein the weight percent is based on the total weigh of the dermatological composition.

The dermatological compositions often include one or more solvents. The solvents may be used to solubilize one or more of the skin-active ingredients and/or to solubilize other components of the composition. In some cases the one or more solvents include at least water. Often, the one or more solvents is a mixture of water and one or more water miscible organic solvents such as a polar volatile solvent. The term “polar volatile solvent” denotes, in the present disclosure, a compound which is liquid at ambient temperature (25° C.), which comprises at least one polar group such as a hydroxyl, ester, ketone, ether or aldehyde group, and which has a vapour pressure greater than 1 mbar at 20° C. Non-limiting examples include lower monoalcohols containing from 1 to 5 carbon atoms such as ethanol and isopropanol, C₃-C₄ ketones, C₂-C₄ aldehydes and C₂-C₄ short-chain esters, and mixtures thereof.

The total amount of solvent in the dermatological composition may be from about 25 to about 95 wt. %, about 35 to about 95 wt. %, about 50 to about 95 wt. %, about 60 to about 95 wt. %, about 75 to about 90 wt. %, about 25 to about 90 wt. %, about 35 to about 90 wt. %, about 50 to about 90 wt. %, about 60 to about 90 wt. %, about 75 to about 90 wt. %, about 35 to about 85 wt. %, about 50 to about 85 wt. %, about 60 to about 85 wt. %, or about 75 to about 85 wt. %, wherein the weight percent is based on the total weight of the dermatological composition.

When the dermatological composition is an aqueous composition, the total amount of water in the dermatological composition may be from about 25 to about 95 wt. %, about 35 to about 95 wt. %, about 50 to about 95 wt. %, about 60 to about 95 wt. %, about 75 to about 90 wt. %, about 25 to about 90 wt. %, about 35 to about 90 wt. %, about 50 to about 90 wt. %, about 60 to about 90 wt. %, about 75 to about 90 wt. %, about 35 to about 85 wt. %, about 50 to about 85 wt. %, about 60 to about 85 wt. %, or about 75 to about 85 wt. %, wherein the weight percent is based on the total weight of the dermatological composition. In some cases, one more water-miscible solvents may be included with the water. The total amount of the water-miscible solvents may be about 1 to about 50 wt. %, about 1 to about 25 t. %, about 1 to about 15 wt. %, about 1 to about 10 wt. %, or about 1 to about 5 wt. %, wherein the weight percent is based on the total weight of the dermatological composition.

The instant disclosure further relates to methods of using the integrated under-eye mask and makeup capture device. For example, the instant disclosure relates to methods for treating skin under an eye while simultaneously capturing makeup fallout, the method comprising applying an integrated under-eye mask and makeup capture device under the eye and applying makeup to an area around the eye. During application of the makeup to the area around the eye, the device captures makeup fallout. The method for treating the skin may comprise hydrating the skin, reducing the appearance of wrinkles, reducing the appearance of dark circles or discoloration, tightening the skin, plumping the skin, nourishing the skin, and/or soothing the skin.

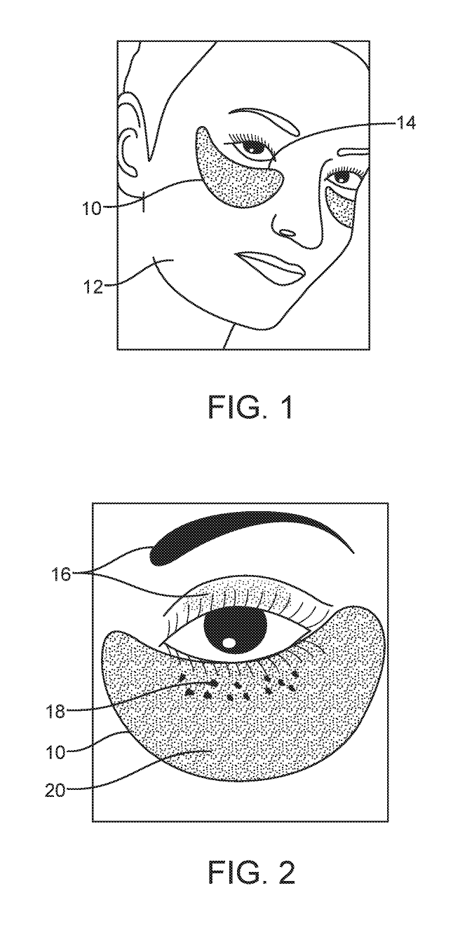

FIG. 1 provides a perspective view of an integrated under-eye mask and makeup capture device (10) applied to the face (12) of a user (under the eye of the user). The upper edge (14) of the integrated under-eye mask and makeup capture device (10) has a concave shape to match the general shape of the eye. FIG. 2 is a front-view of the integrated under-eye mask and makeup capture device (10), which is applied under the eye of a user. Eye makeup (16) appears around the eye. Makeup fallout (particles of makeup) (18) appear on the dry top surface (20) of the integrated under-eye mask and makeup capture device (10).

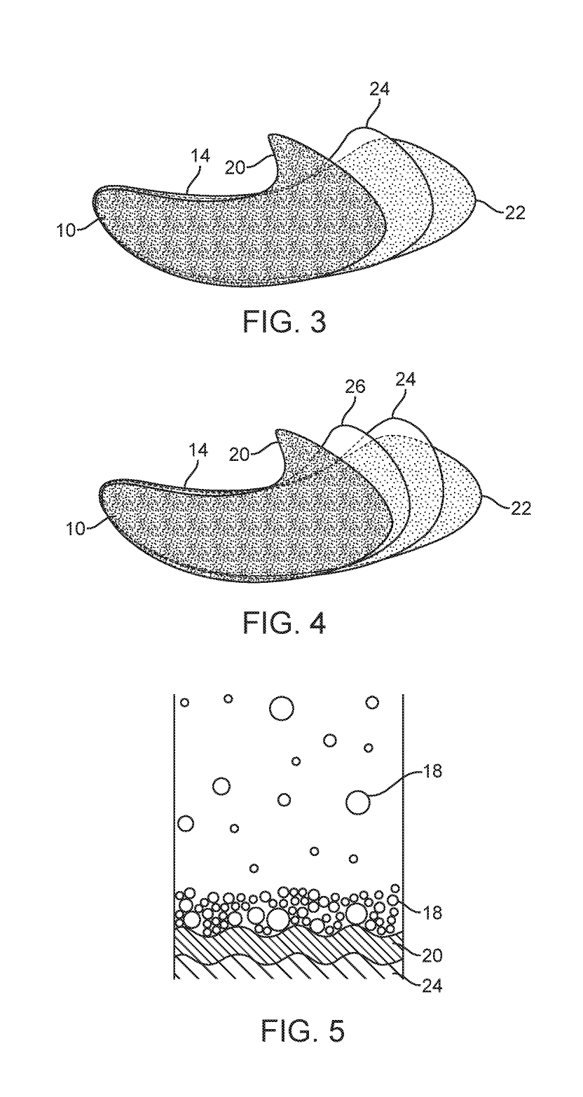

FIG. 3 is and FIG. 4 are both partial side views of integrated under-eye mask and makeup capture device (10) comprising multiple layers. Intermediate layers appear between a dry top surface layer (20) and a moist bottom surface layer (22). For example, in FIG. 3, the integrated under-eye mask and makeup capture device (10) includes an inner liquid resistant layer (24), which may optionally include an adhesive on one or both sides in order to bind or secure the multiple layers into a single composite structure. The adhesive may extend across the entirety of one or both sides of the inner liquid resistant layer (24) or may only extend partial across the one or both sides of the inner liquid resistant layer (24), for example, around the other edges (along the perimeter). The multiple layers may be bound together by methods other than the use of an adhesive. For example, the layers maybe stitched, compressed, or stapled together, etc. Additionally, the device may be assembled at the time of use. For example, a user may apply a moist bottom layer (22) and optionally, subsequently apply additional layers (e.g., an adhesive layer (26), an inner liquid resistant layer (24), a top surface layer (20), etc. In any event, the integrated under-eye mask and makeup capture device (10) (and its layers) are flexible.

FIG. 4 includes a double sided adhesive layer (26). It is shown between the dry top surface layer (20) and the inner liquid resistant layer (24), but can be included elsewhere. For example, it can alternatively or additionally be placed between the moist bottom surface layer (22) and the inner liquid resistant layer (24).

FIG. 4 is a close-up view of the dry top surface layer (20) and the underlying liquid resistant layer (24). Makeup fallout (18) is shown falling onto the dry top surface layer (20). The dry top surface layer (20) attracts the makeup fallout (18) due to electrostatic charge. The dry top surface layer (20) can be post-treated to impart an electrostatic charge or can be made from material that is already electrostatically charged. For example, the dry top surface layer (20) maybe comprise electrostatic fibers.

Implementation of the present disclosure is provided by way of the following examples. The examples serve to illustrate the technology without being limiting in nature.

### Example 1

### Dermatological Composition

A dermatological composition useable in the integrated under-eye mask and makeup capture device described herein is presented in the table below.

| Ingredient | | Concentration |
|---|---|---|
| (INCI US Name) | Characterization | (wt. %) |
| GLYCERIN & | Polylol | 4 |
| BUTYLENE GLYCOL | | |
| NIACINAMIDE (B₃), | Skin-Active | 4.1 |
| SODIUM | Ingredients | |
| HYALURONATE, & | | |
| LACTIC ACID | | |
| CHLORPHENESIN, | Preservatives | 0.6 |
| PHENOXYETHANOL | | |
| SODIUM, & | | |
| BENZOATE | | |
| Emollients, Surfactants, Antioxidants, | | 6 |
| and Conditioning Agents | | |
| Fragrance(s) | | <1% |
| ALCOHOL | Solvent/Preservative | 3 |
| WATER | Solvent | q.s. |

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular.

The expression “at least one” means one or more and thus includes individual components as well as mixtures/combinations.

All ranges and values disclosed herein are inclusive and combinable. For examples, any value or point described herein that falls within a range described herein can serve as a minimum or maximum value to derive a sub-range, etc.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. An integrated under-eye mask and makeup capture device comprising:
a deformable sheet-like substrate having:
a dry top surface for capturing makeup fallout;
a moist bottom surface for application to the skin; and
a upper edge to fit under an eye of a user;
wherein the bottom moist surface is impregnated with a dermatological composition comprising one or more skin-active ingredients.

2. The integrated under-eye mask and makeup capture device of claim 1, wherein the dry top surface is electrostatically charged.

3. The integrated under-eye mask and makeup capture device of claim 1, wherein the bottom moist surface does not include an adhesive that comes into contact with the skin.

4. The integrated under-eye mask and makeup capture device of claim 1, wherein the dermatological composition is an emulsion or an aqueous dispersion.

5. The integrated under-eye mask and makeup capture device of claim 1, wherein the one or more skin-active ingredients are selected from the group consisting of a wrinkle reducing ingredient, a skin whitening ingredient, a moisturizing ingredient, a keratolytic agent, a desquamating agent, a keratinocyte proliferation enhancer, a collagenase inhibitor, an elastase inhibitor, an anti-inflammatory agent, a steroid, an anti-acne agent, and/or an antioxidant.

6. The integrated under-eye mask and makeup capture device of claim 1, wherein the one or more skin-active ingredients are selected from the group consisting of sodium hyaluronic acid, a peptide, linseed extract, a rice protein, caffeine, hesperetin laurate, proxylane, ascorbyl glucoside, and optical effect pigments.

7. The integrated under-eye mask and makeup capture device of claim 2, wherein the deformable sheet-like substrate comprises electrostatic fibers.

8. The integrated under-eye mask and makeup capture device of claim 7, wherein the electrostatic fibers comprise polypropylene and acrylic fibers, or triboelectric fibers.

9. The integrated under-eye mask and makeup capture device of claim 1, wherein the deformable sheet-like substrate comprises wood fibers.

10. The integrated under-eye mask and makeup capture device of claim 1, wherein the deformable sheet-like substrate comprises at least two layers and the dry top surface is formed on a separate layer than the moist bottom surface.

11. The integrated under-eye mask and makeup capture device of claim 10, wherein a waterproof layer lies between the layer comprising the top dry surface and the layer comprising the bottom moist surface, thereby preventing the dermatological composition of the bottom moist surface from reaching the dry top surface.

12. The integrated under-eye mask and makeup capture device of claim 1, wherein the dermatological composition comprises:
(a) one or more skin-active ingredients;
(b) optionally, one or more polyols;
(c) one or more preservatives; and
(d) one or more solvents.

13. The integrated under-eye mask and makeup capture device of claim 12, wherein the one or more skin-active ingredients of (a) is/are selected from the group consisting of sodium hyaluronic acid, niacinamide, a peptide, linseed extract, a rice protein, caffeine, hesperetin laurate, ceramides (e.g., ceramide-3 and/or ceramide-EOP), cholesterol, proxylane, and ascorbyl glucoside.

14. The integrated under-eye mask and makeup capture device of claim 12, wherein the one or more polyols of (b) is/are selected from the group consisting of glycerol, propylene glycol, butylene glycol, hexylene glycol, a polyethylene glycol, and dipropylene glycol.

15. The integrated under-eye mask and makeup capture device of claim 12, wherein the one or more preservatives of (c) is/are selected from the group consisting of phenoxyethanol, benzyl alcohol, salicylic acid, sodium benzoate, benzoic acid, caprylyl glycol, methyl paraben, propyl paraben, ethylhexylglycerin, 1,3-propanediol, cholorphensin, methylchloroisothiazolinone, methylisothiazolinone, benzalkonium chloride, polyaminopropyl biguanide, and glyceryrrihia glabra.

16. The integrated under-eye mask and makeup capture device of claim 12, wherein the one or more solvents of (d) comprises water.

17. A method for treating skin under an eye while simultaneously capturing makeup fallout comprising applying an integrated under-eye mask and makeup capture device of claim 1 under the eye and applying makeup to an area around the eye.

18. The method of claim 13, wherein treating the skin under the eye comprises hydrating the skin.

19. The method of claim 13, wherein treating the skin under the eye comprises reducing the appearance of wrinkles.

20. The method of claim 13, wherein treating the skin under the eye comprises reducing the appearance of dark circles or discoloration.
